(19) United States
(12) Patent Application Publication
(10) Pub. No.: US 2013/0310645 A1
(43) Pub. Date: Nov. 21, 2013

(54) OPTICAL SENSING FOR RELATIVE TRACKING OF ENDOSCOPES

(71) Applicants: Adrien Emmanuel Desjardins, Waterloo (CA); Gert Wim 'T Hooft, Eindhoven (NL); Maya Ella Barley, Walton On Thames (GB); Luis Felipe Gutierrez, Jersey City, NJ (US); Raymond Chan, San Diego, CA (US); Robert Manzke, Sleepy Hollow, NY (US)

(72) Inventors: Adrien Emmanuel Desjardins, Waterloo (CA); Gert Wim 'T Hooft, Eindhoven (NL); Maya Ella Barley, Walton On Thames (GB); Luis Felipe Gutierrez, Jersey City, NJ (US); Raymond Chan, San Diego, CA (US); Robert Manzke, Sleepy Hollow, NY (US)

(73) Assignee: KONINKLIJKE PHILIPS N.V., EINDHOVEN (NL)

(21) Appl. No.: 13/982,279

(22) PCT Filed: Jan. 10, 2012
(86) PCT No.: PCT/IB2012/050112

§ 371 Date: Jul. 29, 2013

Related U.S. Application Data
(60) Provisional application No. 61/437,387, filed on Jan. 28, 2011.

(51) Int. Cl. A61B 1/012; A61B 1/267

(52) U.S. Cl. CPC A61B 1/0125; A61B 1/267
USPC 600/113

(57) ABSTRACT

A telescopic endoscope employing a primary endoscope (30, 50) having a instrument channel, a miniature secondary endoscope (40, 60) deployed within the instrument channel of the primary endoscope (30, 50), and an endoscope tracker including one or more sensors (32, 61) and one or markers (41, 52) for sensing any portion of the miniature secondary endoscope (40, 60) extending from a distal end of the instrument channel of the primary endoscope (30, 50).

The present invention generally relates to a relative tracking of a telescopic endoscope having a miniature secondary endoscope deployed within an instrument channel of a larger primary endoscope. The present invention specifically relates to an integrated tracking of both the primary and secondary endoscopes to minimize the position errors that may occur with an individual optical tracking of the miniature secondary endoscope.

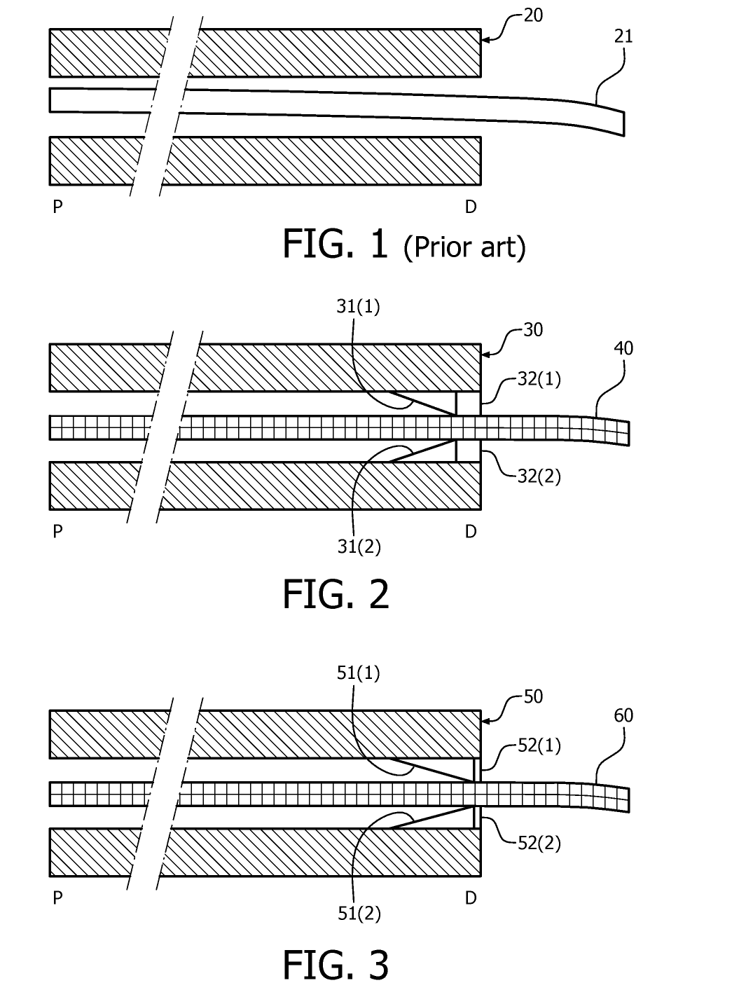

Access to distal regions of the lung is often necessary to perform a biopsy. For endoscopic access to regions that are more distal than the fifth (5^{th}) to sixth (6^{th}) branchpoint of a bronchial tree, a miniature secondary may be used where the miniature secondary endoscope is typically deployed through the instrument channel of a larger primary endoscope. For example, FIG. 1 illustrates a miniature secondary endoscope 21 deployed within a primary endoscope 20 whereby miniature secondary endoscope 21 may be extended to a desired degree from a distal end D of primary endoscope 20.

A significant problem faced by physicians with a miniature secondary endoscope is determining the position of the distal end of the miniature secondary endoscope in the bronchial tree relative to the known anatomy (e.g., anatomy imaged on a pre-procedural CT scan). Tracking the position of endoscopes in real-time is a solution to this problem. Prior art in endoscope tracking has been performed with several methods, including electromagnetic systems and optical fiber shape sensors (e.g., Fiber Bragg Gratings and Rayleigh scattering).

Optical fiber-based shape sensors have many advantages over other tracking methods like electromagnetic tracking. However, one limitation of optical fiber-based shape sensors is achieving high accuracy may be very challenging with very long, flexible probes, particularly those that allow for a significant amount of torsion. Specifically, position errors are known to accrue quadratically with length. Consequently, accurate position tracking of a flexible miniature secondary endoscope with optical fiber shape sensors is significantly more challenging than tracking a traditional primary endoscope that is larger and less flexible. For example, as shown in FIG. 1, accurate position tracking of flexible miniature secondary endoscope 21 with optical fiber shape sensors is significantly more challenging than tracking primary endoscope 20 that is larger and less flexible.

The present invention provides a technique of simultaneously tracking a larger primary endoscope and a miniature secondary endoscope with optical fiber sensing, so that position errors that arise with individually tracking the miniature secondary endoscope may be minimized. Furthermore, a multi-core fiberscope may serve as the miniature secondary endoscope whereby individual pixel fibers of the multi-core fiberscope may be used for shape sensing interrogation using Rayleigh scatter reflection patterns.

For purposes of the present invention, the terms “primary” and “miniature secondary” are not intended to specify any particular dimensions of the devices being described by these terms. The actual use of the terms is to differentiate the relative dimensions of the devices being described by these terms.

One form of the present invention is a telescopic endoscope including a primary endoscope, miniature secondary endoscope and an endoscope tracker. The primary endoscope has an instrument channel, the miniature secondary endoscope is deployed within the instrument channel of the primary endoscope, and the endoscope tracker includes one or more sensors and one or more markers for sensing any portion of the miniature secondary endoscope extending from a distal end of the instrument channel of the primary endoscope.

A second form of the present invention is an optical tracking method involving a deployment of the miniature secondary endoscope within an instrument channel of the primary endoscope, and an operation of the endoscope tracker for sensing any portion of the miniature secondary endoscope extending from a distal end of the instrument channel of the primary endoscope.

The foregoing forms and other forms of the present invention as well as various features and advantages of the present invention will become further apparent from the following detailed description of various exemplary embodiments of the present invention read in conjunction with the accompanying drawings. The detailed description and drawings are merely illustrative of the present invention rather than limiting, the scope of the present invention being defined by the appended claims and equivalents thereof

FIG. 1 illustrates a side view of an exemplary embodiment of a telescopic endoscope as known in the art.

FIGS. 2 and 3 illustrate side views of exemplary embodiments of telescopic endoscopes in accordance with the present invention.

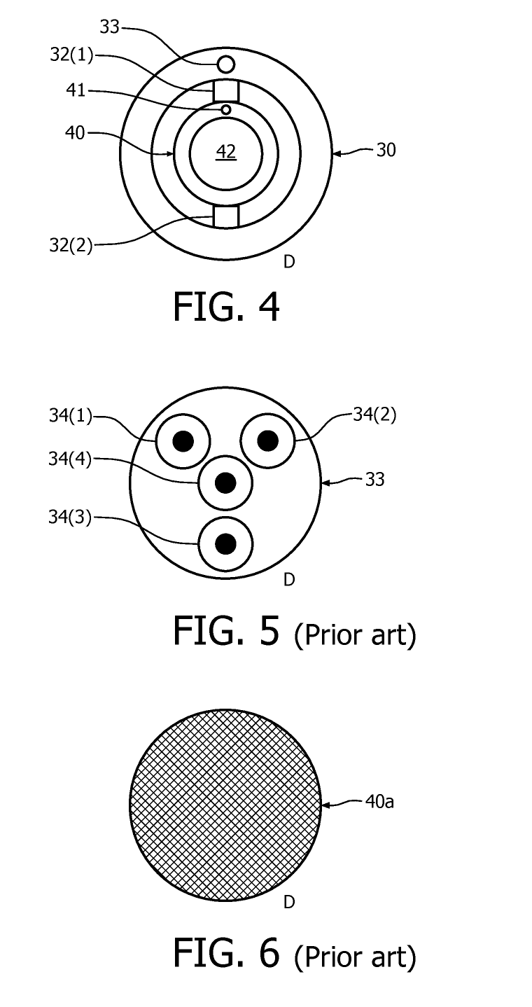

FIG. 4 illustrates a distal end view of the telescopic endoscope shown in FIG. 2.

FIG. 5 illustrates a distal end view of an exemplary embodiment of an optical fiber as known in the art.

FIG. 6 illustrates a distal end view of an exemplary embodiment of a fiberscope as known in the art.

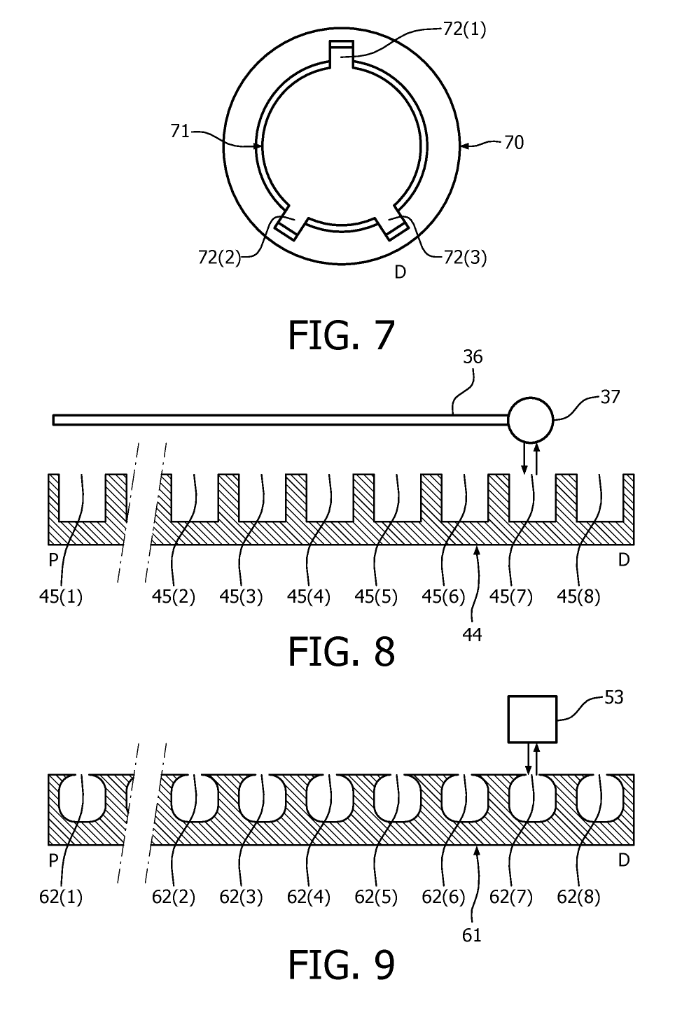

FIG. 7 illustrates a distal end view of an exemplary embodiment of a third exemplary embodiment of a telescopic endoscope in accordance with the present invention.

FIGS. 8 and 9 illustrate side views of an exemplary embodiment of endoscope trackers respectively shown in FIGS. 2 and 3.

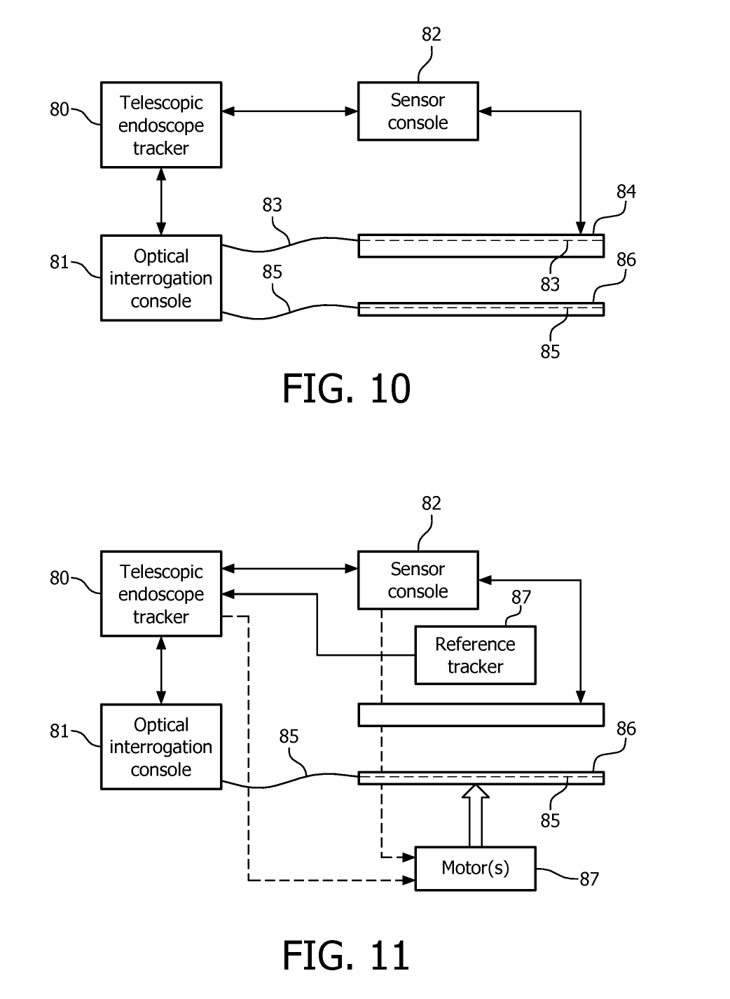

FIGS. 10 and 11 illustrate exemplary embodiments of optical tracking system in accordance with the present invention.

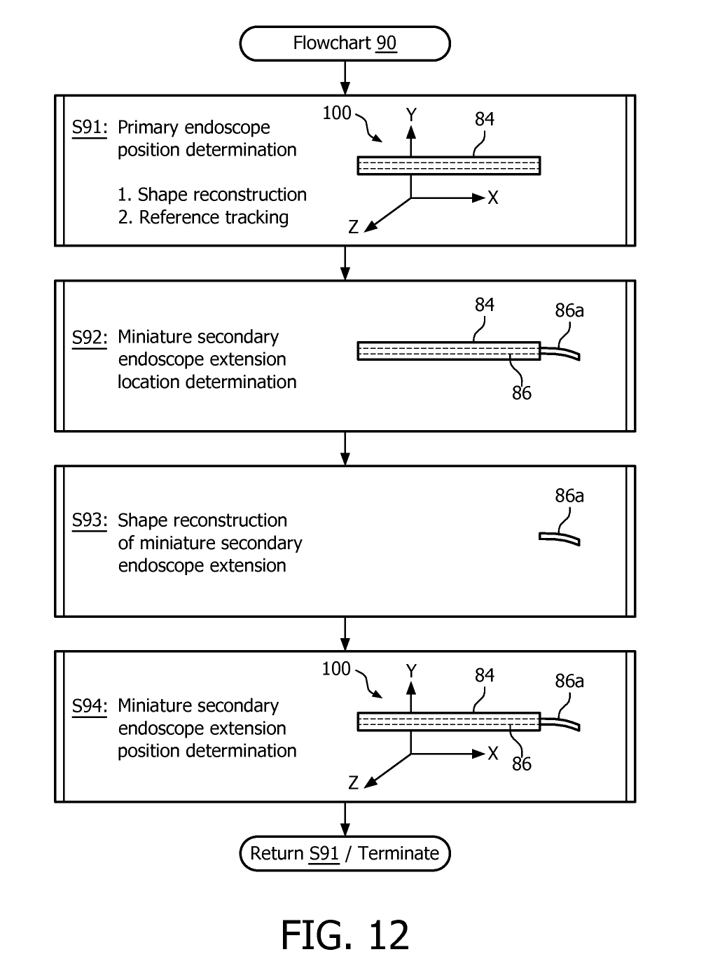

FIG. 12 illustrates a flowchart representative of an optical tracking method of a telescopic endoscope in accordance with the present invention.

As shown in FIG. 2, one embodiment of a telescopic endoscope of the present invention employs a primary endoscope 30 and a miniature secondary endoscope 40 deployed within an instrument channel of primary endoscope 30. The telescopic endoscope further employs a secondary endoscope tracker including two (2) sensors 32 and a plurality of markers axially aligned along miniature secondary endoscope 40 as indicated by the hatched lines through miniature secondary endoscope 40.

As will be further explained herein in connection with the description of FIGS. 10-12, the present invention is premised on tracking a portion of miniature secondary endoscope 40 extending from a distal end D of the instrument channel of primary endoscope 30 as opposed to tracking the entire miniature secondary endoscope 40. Thus, as miniature secondary endoscope 40 is translated within primary endoscope 30 in either a proximal P direction or a distal direction D, sensors 32 sense the portion of miniature secondary endoscope 40 extending from the distal end D of the instrument channel of primary endoscope 30 at any given moment via a systematic sensing of the markers to thereby facilitate the extended portion tracking of the miniature secondary endoscope 40. Sensors 32 may also sense an angular orientation of the miniature secondary endoscope 40 relative to the distal end D of the instrument channel of primary endoscope 30 via the markers to further facilitate the extended portion tracking of miniature secondary endoscope 40.

In one embodiment, the markers are disposed at regular intervals along the length of miniature secondary endoscope 40 whereby sensors 32 count how many markers have passed by as the miniature secondary endoscope 40 is translated within primary endoscope 30 in either a proximal P direction (−) or a distal direction D (+) to thereby determine the extended portion of miniature secondary endoscope 40. Additionally, the markers at different angles are differently colored whereby an angle of miniature secondary endoscope 40 is sensed by how the differently-colored markings are oriented relative to the distal end D of the instrument channel of primary endoscope 30.

The telescopic endoscope of FIG. 2 further employs two (2) guides 31 for controlling a position and angulation of the extended portion of miniature secondary endoscope 40 to assist in the sensing of the of the extended portion of miniature secondary endoscope 40.

As shown in FIG. 3, one alternative embodiment of a telescopic endoscope of the present invention employs a primary endoscope 50 and a miniature secondary endoscope 60 deployed within an instrument channel of primary endoscope 50. The telescopic endoscope further employs a secondary endoscope tracker including two (2) markers 52 and a plurality of sensors axially aligned along miniature secondary endoscope 60 as indicated by the hatched lines through miniature secondary endoscope 60.

Again, as will be further explained herein in connection with the description of FIGS. 10-12, the present invention is premised on tracking a portion of miniature secondary endoscope 60 extending from a distal end D of the instrument channel of primary endoscope 50 as opposed to tracking the entire miniature secondary endoscope 60. Thus, as miniature secondary endoscope 60 is translated within primary endoscope 50 in either a proximal P direction or a distal direction D, the sensors of miniature secondary endoscope 60 sense the portion of miniature secondary endoscope 60 extending from the distal end D of the instrument channel of primary endoscope 50 at any given moment via a systematic sensing of markers 52 as known in the art to thereby facilitate the extended portion tracking of the miniature secondary endoscope 60. The sensors of miniature secondary endoscope 60 may also sense an angular orientation of the miniature secondary endoscope 60 relative to the distal end D of the instrument channel of primary endoscope 30 via the markers 52 to further facilitate the extended portion tracking of miniature secondary endoscope 60.

In one embodiment, the sensors are disposed at regular intervals along the length of miniature secondary endoscope 60 whereby each sensor passing by multi-colored markers 52 as the miniature secondary endoscope 60 is translated within primary endoscope 50 in either a proximal P direction (−) or a distal direction D (+) is counted to thereby determine the extended portion of miniature secondary endoscope 50. Additionally, sensors at different angles may provide differing color filters whereby an angle of miniature secondary endoscope 60 is sensed by how the differing color filters are oriented relative to the distal end D of the instrument channel of primary endoscope 50.

The telescopic endoscope of FIG. 3 further employs two (2) guides 51 for controlling a position and angulation of the extended portion of miniature secondary endoscope 60 to assist in the sensing of the of the extended portion of miniature secondary endoscope 60.

The tracking of an extended portion of a miniature secondary endoscope is based on an optical shape sensing of the miniature secondary endoscope, and an optical shape sensing or reference tracking of a primary endoscope as will be further explained in connection with the description of FIGS. 10-12. For example, as shown in FIG. 4, primary endoscope 30 (FIG. 2) may include an optical fiber 33 deployed within a tracking channel of primary endoscope 30, and miniature secondary endoscope 40 may include an optical fiber 41 deployed within a tracking channel of miniature secondary endoscope 40.

For purposes of the present invention, the term “optical fiber” is broadly defined herein as any article or device structurally configured for transmitting/reflecting light by means of successive internal optical reflections via a deformation sensor array with each deformation optic sensor of array being broadly defined herein as any article structurally configured for reflecting a particular wavelength of light while transmitting all other wavelengths of light whereby the reflection wavelength may be shifted as a function of an external stimulus applied to the optical fiber. Examples of optical fiber include, but are not limited to, a flexible optically transparent glass or plastic fiber incorporating an array of fiber Bragg gratings integrated along a length of the fiber as known in the art, and a flexible optically transparent glass or plastic fiber having naturally variations in its optic refractive index occurring along a length of the fiber as known in the art (e.g., a Rayleigh scattering based optical fiber).

In practice, each optical fiber may include one or more fiber cores as known in the art, such as, for example, a multi-core embodiment of optical fiber 33 having a known helical arrangement of four (4) cores 34 as shown in FIG. 5.

Referring back to FIGS. 1 and 2, miniature secondary endoscopes 40, 60 may include an imaging channel and an optical fiber as shown in FIG. 3 or alternatively, may be fiberscopes as known in the art. For example, FIG. 6 shows a fiberscope version 40a of miniature secondary endoscope 40. An advantage of this version 40a is the fiberscope may serve as both an imaging fiber as known in the art and as an optical shape sensor based on an inherent characteristic Rayleigh scatter pattern of the fiberscope.

As shown in FIG. 7, an axial alignment of a primary endoscope 70 and a miniature secondary endoscope 71 provide an alternative to the use of guides (e.g., guides 31 of FIG. 2 and guides 51 of FIG. 3) for controlling a position and an angulation of the extended portion of a miniature secondary endoscope. In this alternative embodiment, three (3) protrusions 72 of miniature secondary endoscope 71 are slidably inserted within grooves of primary endoscope 70 to axial align the endoscopes. Alternatively, primary endoscope 70 may have protrusions slidably inserted within grooves of miniature secondary endoscope 71.

In practice, the sensors and the markers of the secondary endoscope tracker may be based on any physical parameter suitable for sensing the extended portion of a miniature secondary endoscope. For example, the endoscope tracker may utilize an optical color sensing as previously described herein, a magnetic sensing, an electrical capacitance sensing, an impedance sensing, a field strength sensing, a frequency sensing, an acoustic sensing, a chemical sensing and other sensing techniques as well known in the art.

FIG. 8 illustrates an alternative optical sensing having a sensor constructed with an optical fiber 36 and a ball-lens 37 having a polished tip for delivering broadband focused light to markers 45 of a miniature secondary endoscope 44, which is reflected back to lens 37. The reflected light is spectrally processed to determine a dominant color that is reflected from markers 45. Given that different angular positions on the sensors have different colors, the dominant color reveals the angle of miniature secondary endoscope 44 inside the instrument channel of a primary endoscope. This position/angle sensor has the advantage that it does not require electrical current to be delivered to the tip. Furthermore, alternative types of marking schemes on the miniature endoscope may be implemented (e.g., black-and-white markers or gray-scale markers).

FIG. 9 illustrates an alternative optical sensing having multiple optic fibers 62 delivering light from the surface of a miniature secondary endoscope 61 whereby light reflected back by a marker 53 facilitates the optical sensing of miniature secondary endoscope 61.

As description of an optical tracking system and method will now be provided herein to facilitate a further understanding of the present invention.

As shown in FIG. 10, an optical tracking system of the present invention employs a telescopic endoscope tracker 80, an optical interrogation console 81 and a sensor console 82, an optic fiber 83 deployed within a primary endoscope 84, and an optic fiber 85 deployed within a miniature secondary endoscope 86.

Telescopic endoscope tracker 80 is broadly defined herein as any device or system structurally configured for executing a shape reconstruction algorithm for reconstructing a shape of optical fiber 83 and/or optical fiber 85 as will be further explained with the description of FIG. 12.

Optical interrogation console 81 is broadly defined herein as any device or system structurally configured for transmitting light through optical fibers 83 and 85 for processing encoded optical signals of reflection spectrums generated by the successive internal reflections of the transmitted light via the deformation optic sensor arrays of optical fibers 83 and 85. In one embodiment, optical interrogation console 81 employs an arrangement (not shown) of a coherent optical source, a frequency domain reflectometer, and other appropriate electronics/devices as known in the art.

Sensor console 82 is broadly defined herein as any device or system structurally configured for executing a sensing algorithm appropriate for the sensing scheme being implemented by the secondary endoscope tracker of sensors and markers.

Collectively, telescopic endoscope tracker 80, an optical interrogation console 81 and a sensor console 82 implement a flowchart 90 (FIG. 12) for tracking endoscopes 84 and 86.

Referring to FIG. 12, optical fibers 83 and 85 are registered with a tracking coordinate system 100 associated with the system.

A stage S91 of flowchart 90 encompasses a determination of a position of a primary endoscope 84 within tracking coordinate system 100 of console 81, particularly a position of distal end of primary endoscope 84 within tracking coordinate system 100. Specifically, optical interrogation console 81 operates optical fiber 83 to thereby facilitate a reconstruction of a shape of primary endoscope 84 by telescopic endoscope tracker 80.

A stage S92 of flowchart 90 encompasses a determination of any extended portion 86a of miniature secondary endoscope 86. Specifically, sensor console 82 operates the sensors of the secondary endoscope tracker as previously taught herein to thereby determine extended portion 86a.

A stage S93 of flowchart 90 encompasses a reconstruction of a shape of the extended portion 86a of miniature secondary endoscope 86. Specifically, optical interrogation console 81 operates optical fiber 85 to thereby facilitate a reconstruction of extended endoscope portion 86a by telescopic endoscope tracker 80 as sensed by sensor console 82.

A stage S94 of flowchart 90 encompasses a determination of a position of extended portion 86a within optical coordinate system 100 relative to the distal end of primary endoscope 84 by telescopic endoscope tracker 80.

Stages S91-S94 are repeated as many as necessary until the tracking of endoscopes 84 and 86 is terminated.

Referring to FIG. 11, an alternative optical tracking system of the present invention employs a reference tracker 87 and optionally employs one or more motors 88.

Reference tracker 87 is broadly defined herein as any type of device or system for tracking endoscope or the like within a reference coordinate system. Examples of reference tracker 87 include, but are not limited to, an electromagnetic tracking system, an optical tracking system and an imaging tracking system. With this embodiment, the determination of a position of endoscope 84 within a reference coordinate system during stage S91 (FIG. 12) is performed by reference tracker 87 and communicated to telescopic endoscope tracker 80. The optical shape sensing system coordinate system 100 is registered to the coordinate system of reference tracker 87 and the flowchart 90 proceeds as previously described herein.

Motor(s) 88 may be operated to advance/rotate miniature secondary endoscope 86 beyond/within primary endoscope 84 via mechanical actuation. Preferably, motor(s) 88 operate in accordance with a closed-loop control with feedback from the sensors of the endoscope tracker. Feedback to motor(s) 88 may also be provided from the output of the shape determination algorithm via telescopic endoscope tracker 80. In this way, mechanical control of miniature secondary endoscope 84 may be performed in a semi-automated or fully-automated manner by taking into account structural features identified with pre-procedural or intra-procedural images.

Referring to FIGS. 10 and 11, in practice, a live visualization of endoscope 84 and 86 as a virtual model showing manipulation of the deployment geometry together with the inner miniature endoscope 86 may be implemented. This virtual model will provide instantaneous information as known in the art about configuration, dynamics, error/confidence feedback about position/orientation, superimposed on concurrent imaging, preprocedural information, or other relevant clinical biometrics/bioinformatics.

Still referring to FIGS. 10 and 11, a relative position/motion of endoscopes 84 and 86 endoscope configurations may be used as input gestures as known in the art to trigger (semi)-automated changes in imaging characteristics, configurations, visualization modes, data processing modes, etc.

From the description of FIGS. 2-12, those having ordinary skill in the art will have a further appreciation on how to manufacture and use an optical tracking system for any kind of telescopic device having two or more elongated devices in accordance with the present invention for numerous surgical procedures. Examples of the elongated devices include, but are not limited to, endoscopes, catheters and guidewires.

While various exemplary embodiments of the present invention have been illustrated and described, it will be understood by those skilled in the art that the exemplary embodiments of the present invention as described herein are illustrative, and various changes and modifications may be made and equivalents may be substituted for elements thereof without departing from the true scope of the present invention. For example, although the invention is discussed herein with regard to FBGs, it is understood to include fiber optics for shape sensing or localization generally, including, for example, with or without the presence of FBGs or other optics, sensing or localization from detection of variation in one or more sections in a fiber using back scattering, optical fiber force sensing, fiber location sensors or Rayleigh scattering. In addition, many modifications may be made to adapt the teachings of the present invention without departing from its central scope. Therefore, it is intended that the present invention not be limited to the particular embodiments disclosed as the best mode contemplated for carrying out the present invention, but that the present invention includes all embodiments falling within the scope of the appended claims.

1. A telescopic endoscope, comprising:
a primary endoscope (30, 50) having a instrument channel;
a miniature secondary endoscope (40, 60) deployed within the instrument channel of the primary endoscope (30, 50); and
an endoscope tracker including at least one sensor and at least one marker for sensing any portion of the miniature secondary endoscope (40, 60) extending from a distal end of the instrument channel of the primary endoscope (30, 50).

2. The telescopic endoscope of claim 1,
wherein the at least one sensor (32, 52) is integrated with the primary endoscope (30, 50); and
wherein the at least one marker (43) is integrated with the miniature secondary endoscope (40, 60).

3. The telescopic endoscope of claim 1,
wherein the at least one marker (52) is integrated with the primary endoscope (30, 50); and
wherein the at least one sensor (61) is integrated with the miniature secondary endoscope (40, 60).

4. The telescopic endoscope of claim 1, wherein the endoscope tracker is further operable for sensing an angular orientation of any extended portion of the miniature secondary endoscope (40, 60).

5. The telescopic endoscope of claim 1, wherein the primary endoscope (30, 50) includes at least one optical fiber for reconstructing a shape of the primary endoscope (30, 50).

6. The telescopic endoscope of claim 1, wherein the secondary endoscope (40, 60) includes at least one optical fiber for reconstructing a shape of any extended portion of the miniature secondary endoscope (40, 60) sensed by the endoscope tracker.

7. The telescopic endoscope of claim 1, wherein the miniature secondary endoscope (40, 60) is a fiberscope (42a).

8. The telescopic endoscope of claim 7, wherein the fiberscope (42a) is operable for reconstructing a shape of any extended portion of the miniature secondary endoscope (40, 60) sensed by the endoscope tracker.

9. The telescopic endoscope of claim 1, wherein the primary endoscope (30, 50) includes at least one guide (31, 51) for defining an exit opening at the distal end of the instrument channel of the primary endoscope.

10. The telescopic endoscope of claim 1, wherein the miniature secondary endoscope (40, 60) is axially aligned within the instrument channel of the primary endoscope (30, 50).

11. An optical tracking system, comprising:
a telescopic endoscope including
a primary endoscope (30, 50) having a instrument channel,
a miniature secondary endoscope (40, 60) deployed within the instrument channel of the primary endoscope (30, 50), and
an endoscope tracker includes at least one sensor and at least one marker for sensing any portion of the miniature secondary endoscope (40, 60) extending from a distal end of the instrument channel of the primary endoscope (30, 50); and
a sensor console (82) in communication with the at least one sensor for monitoring the sensing of any extended portion of the miniature secondary endoscope (40, 60).

12. The optical tracking system of claim 11, further comprising:
a telescopic endoscope tracker (80) and an optical interrogation console (81),
wherein the miniature secondary endoscope (40, 60) includes at least one secondary optical fiber,
wherein the optical interrogation console (81) is in communication with the at least one secondary optical fiber for sensing a shape of the miniature secondary endoscope (40, 60), and
wherein the telescopic endoscope tracker (80) is in communication with the sensor console (82) and the optical interrogation console (81) for reconstructing a shape of any extended portion of the miniature secondary endoscope (40, 60) as sensed by the endoscope tracker and the optical interrogation console (81).

13. The optical tracking system of claim 12,
wherein the primary endoscope (30, 50) includes at least one primary optical fiber;
wherein the optical interrogation console (81) is in communication with the at least one primary optical fiber for sensing a shape of the primary endoscope (30, 50); and
wherein the telescopic endoscope tracker (80) is in communication with the optical interrogation console (81) for reconstructing the shape of the primary endoscope (30, 50) as sensed by the optical interrogation console (81).

14. The optical tracking system of claim 13,
wherein the telescopic endoscope tracker (80) is operable to determine a position of the primary endoscope (30, 50) within a tracking coordinate system as a function of a shape reconstruction of the primary endoscope (30, 50); and
wherein the telescopic endoscope tracker (80) is further operable to determine a position of the miniature secondary endoscope (40, 60) relative to the distal end of the primary endoscope (30, 50) within the tracking coordinate system as a function of a shape reconstruction of any extended portion of the miniature secondary endoscope (40, 60) relative to the shape reconstruction of the primary endoscope (30, 50).

15. The optical tracking system of claim 12, further comprising:
a reference tracker (83) for determining a position of the primary endoscope (30, 50) within a tracking coordinate system,
wherein the telescopic endoscope tracker (80) is in communication with the reference tracker (83) for determining a position of the miniature secondary endoscope (40, 60) relative to the distal end of the primary endoscope (30, 50) within the tracking coordinate system as a function of a shape reconstruction of any extended portion of the miniature secondary endoscope (40, 60) relative to the location of the primary endoscope (30, 50) within the tracking coordinate system.

16. An optical tracking method for a telescopic endoscope including a primary endoscope (30, 50), a miniature secondary endoscope (40, 60) and an endoscope tracker, the optical tracking method comprising:
deploying the miniature secondary endoscope (40, 60) within a instrument channel of the primary endoscope (30, 50); and
operating the endoscope tracker for sensing any portion of the miniature secondary endoscope (40, 60) extending from a distal end of the instrument channel of the primary endoscope (30, 50).

17. The optical tracking method of claim 16, further comprising:
sensing a shape of the miniature secondary endoscope (40, 60); and
reconstructing a shape of any extended portion of the miniature secondary endoscope (40, 60) as sensed by the endoscope tracker.

18. The optical tracking method of claim 17, further comprising:
sensing a shape of the primary endoscope (30, 50); and
reconstructing a shape of the primary endoscope (30, 50).

19. The optical tracking method of claim 18, further comprising:
determining a position of the primary endoscope (30, 50) within a tracking coordinate system as a function of a shape reconstruction of the primary endoscope (30, 50); and
determining a position of the miniature secondary endoscope (40, 60) relative to the distal end of the primary endoscope (30, 50) within the tracking coordinate system as a function of a shape reconstruction of any extended portion of the miniature secondary endoscope (40, 60) relative to the shape reconstruction of the primary endoscope (30, 50).

20. The optical tracking method of claim 17, further comprising:
determining a position of the primary endoscope (30, 50) within a tracking coordinate system as a function of a reference tracking; and
determining a position of the miniature secondary endoscope (40, 60) relative to the distal end of the primary endoscope (30, 50) within the tracking coordinate system as a function of a shape reconstruction of any extended portion of the miniature secondary endoscope (40, 60) relative to the determined location of the primary endoscope (30, 50) within the tracking coordinate system.
